(19) United States
(12) Patent Application Publication
(10) Pub. No.: US 2023/0081198 A1
(43) Pub. Date: Mar. 16, 2023

(54) Magnetically Oriented Fiber Optic Three-Dimensional Shape

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 17/945,934

(22) Filed: Sep. 15, 2022

Related U.S. Application Data
(60) Provisional application No. 63/245,015, filed on Sep. 16, 2021.

(51) Int. Cl. A61B 34/20; A61B 5/283; A61B 5/346; A61B 5/00; A61B 5/06

(52) U.S. Cl. CPC A61B 34/20; A61B 5/283; A61B 5/346; A61B 5/6847; A61B 5/062; A61B 2034/2061; A61B 2034/2051; A61B 2562/0266

(57) ABSTRACT

Disclosed herein are systems and methods for providing tracking information of a medical instrument using optical fiber technology in combination with magnetic sensing technology. The medical device includes an optical fiber. The medical device further includes magnetic elements. A magnet field sensor can be configured to detect magnetic fields defined by the magnetic elements, and to provide electrical signals in accordance with the detection of the magnetic fields to a console. The operations of the console include (i) processing the reflected light signals to determine a physical state of the optical fiber, (ii) processing the electrical signals to determine the positions of the magnetic elements, and (iii) combining the physical state of the medical device with the positions of the one or more of the plurality of magnetic elements to determine at least one of a position, a shape, and an orientation of the medical device within the patient body.

### PRIORITY

This application claims the benefit of priority to U.S. Provisional Application No. 63/245,015, filed Sep. 16, 2021, which is incorporated by reference in its entirety into this application.

### BACKGROUND

In the past, certain intravascular guidance of medical devices, such as guidewires and catheters for example, have used fluoroscopic methods for tracking tips of the medical devices and determining whether distal tips are appropriately localized in their target anatomical structures. However, such fluoroscopic methods expose patients and their attending clinicians to harmful X-ray radiation. Moreover, in some cases, the patients are exposed to potentially harmful contrast media needed for the fluoroscopic methods.

More recently, electromagnetic tracking systems have been used involving stylets. Generally, electromagnetic tracking systems feature three components: a field generator, a sensor unit and control unit. The field generator uses several coils to generate a position-varying magnetic field, which is used to establish a coordinate space. Attached to the stylet, such as near a distal end (tip) of the stylet for example, the sensor unit includes small coils in which current is induced via the magnetic field. Based on the electrical properties of each coil, the position and orientation of the medical device may be determined within the coordinate space. The control unit controls the field generator and captures data from the sensor unit.

Disclosed herein is a combination of fiber optic shape sensing system with electromagnetic tracking systems to provide improved guidance and placement confirmation of intravascular device. Some embodiments combine the fiber optic shape sensing functionality with electrocardiogram (ECG) monitoring.

### SUMMARY

Briefly summarized, embodiments disclosed herein are directed to systems, apparatus and methods for providing tracking information of a medical instrument using optical fiber technology in combination with magnetic sensing technology. According to one embodiment, a medical device system for detecting placement of a medical device within a patient body, medical device for insertion within the patient body that includes an optical fiber having one or more of core fibers, each of the one or more core fibers including a plurality of fiber sensors distributed along a longitudinal length of a corresponding core fiber and each fiber sensor of the plurality of fiber sensors being configured to (i) reflect a light signal of a different fiber spectral width based on received incident light, and (ii) change a characteristic of the reflected light signal based on strain experienced by the optical fiber. The medical device further includes a plurality of magnetic elements disposed along a longitudinal length of the medical device, where each magnetic element defines a magnetic field configured to indicate a position of the magnetic element in three-dimensional space. A magnet field sensor is configured to detect one or more magnetic fields defined by one or more of the plurality of magnetic elements, and provides electrical signals in accordance with the detection of the one or more magnetic fields to a console. The magnet field sensor may be coupled to the patient body.

The console includes one or more processors and a non-transitory computer-readable medium having stored thereon logic, when executed by the one or more processors, causes operations of the system. The operations include (i) providing an incident light signal to the optical fiber, (ii) receiving reflected light signals of different spectral widths of the incident light by one or more of the plurality of fiber sensors, (iii) processing the reflected light signals associated with the one or more of core fibers to determine a physical state of the optical fiber. The operations further include (i) receiving the electrical signals from the magnetic field sensor, (ii) processing the electrical signals to determine the positions of the one or more of the plurality of magnetic elements with respect to the magnetic field sensor, and (iii) combining the physical state of the medical device with the positions of the one or more of the plurality of magnetic elements to determine at least one of a position, a shape, and an orientation of the medical device within the patient body.

Each magnetic element may be longitudinally shaped having a magnetic pole at each end, where the magnetic poles define a magnetic field in accordance with an orientation of the magnetic element. In some embodiments, a longitudinal axis of each magnetic element is aligned with a longitudinal axis of the medical device and the operations may further include processing the electrical signals to determine an orientation of each of the magnetic elements with respect to the magnetic field sensor.

According to further embodiments, the operations further include combining the positions of the magnetic elements with the orientations of the magnetic elements to determine at least one of the position, the shape, and the orientation of the medical device within the patient body.

In some embodiments, each magnet is shaped of a hollow cylinder, and the optical fiber is disposed within the hollow cylinder. One of the magnetic elements may be disposed at a distal tip of the medical device and the magnetic field sensor may be applied to a chest of the patient. One or more of the plurality of the magnetic elements may be permanent magnets and one or more of the plurality of the magnetic elements may be formed of a ferrous material. One or more of the plurality of magnetic elements may also be electro-magnets.

One or more of the plurality of magnetic elements may be configured to sense a magnetic field, and one or more of the plurality of magnetic elements may be configured to selectively define a magnetic field, and sense a magnetic field. In some embodiments, the magnetic field sensor includes one or more magnetic elements configured to define a magnetic field.

The medical device may be one of an introducer wire, a guidewire, a stylet, a stylet within a needle, a needle with the optical fiber inlayed into a cannula of the needle or a catheter with the optical fiber inlayed into one or more walls of the catheter. Each of the plurality of fiber sensors may be a reflective grating, where each reflective grating alters its reflected light signal by applying a wavelength shift dependent on a strain experienced by the reflective grating.

According to further embodiments, the operations further include (i) receiving an ECG signal from an ECG electrode disposed at the distal tip, (ii) processing the ECG signal to determine a position of the electrode within the superior vena cava, and (iii) combining the ECG signal with the physical state of the medical device and the positions of the one or more of the plurality of magnetic elements to determine the position of the medical device within the patient body.

As disclosed herein is a method for detecting placement of a medical device within a patient body. The method includes providing an incident light signal to an optical fiber included within the medical device, wherein the optical fiber includes a one or more of core fibers, and each of the one or more of core fibers includes a plurality of reflective gratings distributed along a longitudinal length of a corresponding core fiber. Each of the plurality of reflective gratings is configured to (i) reflect a light signal of a different spectral width based on received incident light, and (ii) change a characteristic of the reflected light signal based on strain experienced by the optical fiber. The method further includes (i) receiving reflected light signals of different spectral widths of the incident light by one or more of the plurality of fiber sensors, (ii) processing the reflected light signals associated with the one or more of core fibers to define physical state of the optical fiber.

The method also includes (i) receiving electrical signals from the magnetic field sensor applied to the patient, where magnets disposed along the medical device define one or magnetic fields detected by the magnetic field sensor, (ii) processing the electrical signals to determine the positions of the one or more of the plurality of magnetic elements with respect to the magnetic field sensor, and (iii) combining the physical state of the medical device with the positions of the one or more of the plurality of magnetic elements to determine at least one of a position, a shape, and an orientation of the medical device within the patient body.

The optical fiber may be a multi-core optical fiber including a plurality of core fibers, and each of the plurality of fiber sensors may be a reflective grating, where each reflective grating alters its reflected light signal by applying a wavelength shift dependent on a strain experienced by the reflective grating.

Each magnetic element may be longitudinally shaped having a magnetic pole at each end, where the magnetic poles define a magnetic field in accordance with an orientation of the magnetic element. In some embodiments of the method, a longitudinal axis of each magnetic element is aligned with a longitudinal axis of the medical device. The method may further include processing the electrical signals to determine an orientation of each of the magnetic elements with respect to the magnetic field sensor.

The method may further include combining the positions of the magnetic elements with the orientations of the magnetic elements to determine at least one of the position, the shape, and the orientation of the medical device within the patient body.

The method may further include (i) receiving an ECG signal from an ECG electrode disposed at a distal tip of the medical device, (ii) processing the ECG signal to determine a position of the electrode within a superior vena cava of the patient, and (iii) combining the ECG signal with the physical state of the medical device and the positions of the one or more of the plurality of magnetic elements to determine the position of the medical device within the patient body.

In some embodiments of the method, the medical device is one of an introducer wire, a guidewire, a stylet, a stylet within a needle, a needle with the optical fiber inlayed into a cannula of the needle or a catheter with the optical fiber inlayed into one or more walls of the catheter. Further according to some embodiments, each magnet is shaped of a hollow cylinder, and the optical fiber is disposed within the hollow cylinder.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1A is an illustrative embodiment of a medical instrument monitoring system including a medical instrument with optic shape sensing capabilities, in accordance with some embodiments;

FIG. 1B is an alternative illustrative embodiment of the medical instrument monitoring system 100 in accordance with some embodiments;

FIG. 2 is an exemplary embodiment of a structure of a section of the multi-core optical fiber included within the stylet 120 of FIG. 1A in accordance with some embodiments;

FIG. 3A is a first exemplary embodiment of the stylet of FIG. 1A supporting both an optical and electrical signaling in accordance with some embodiments;

FIG. 3B is a cross sectional view of the stylet of FIG. 3A in accordance with some embodiments;

FIG. 4A is a second exemplary embodiment of the stylet of FIG. 1B in accordance with some embodiments;

FIG. 4B is a cross sectional view of the stylet of FIG. 4A in accordance with some embodiments;

FIG. 5A is an elevation view of a first illustrative embodiment of a catheter including integrated tubing, a diametrically disposed septum, and micro-lumens formed within the tubing and septum in accordance with some embodiments;

FIG. 5B is a perspective view of the first illustrative embodiment of the catheter of FIG. 5A including core fibers installed within the micro-lumens in accordance with some embodiments;

FIGS. 6A-6B are flowcharts of the methods of operations conducted by the medical instrument monitoring system of FIGS. 1A-1B to achieve optic three-dimensional shape sensing in accordance with some embodiments;

FIG. 7 is an exemplary embodiment of the medical instrument monitoring system of FIGS. 1A-1B during operation and insertion of the catheter into a patient, in accordance with some embodiments;

FIG. 8A is exemplary illustration of the stylet of FIG. 1A located with a three-dimensional coordinate axis space, in accordance with some embodiments;

FIG. 8B is cross-sectional view of the stylet of FIG. 8A, in accordance with some embodiments;

FIG. 9 is another exemplary embodiment of the magnetic locating system FIGS. 1A-1B, in accordance with some embodiments; and

FIG. 10 illustrates a further embodiment of a medical instrument monitoring system, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random-access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

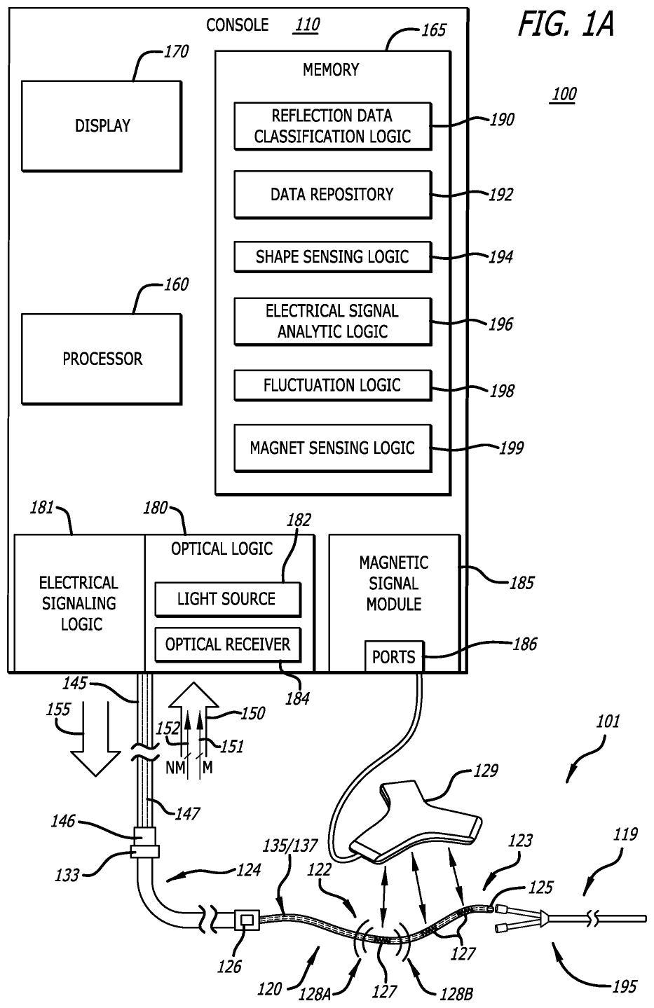

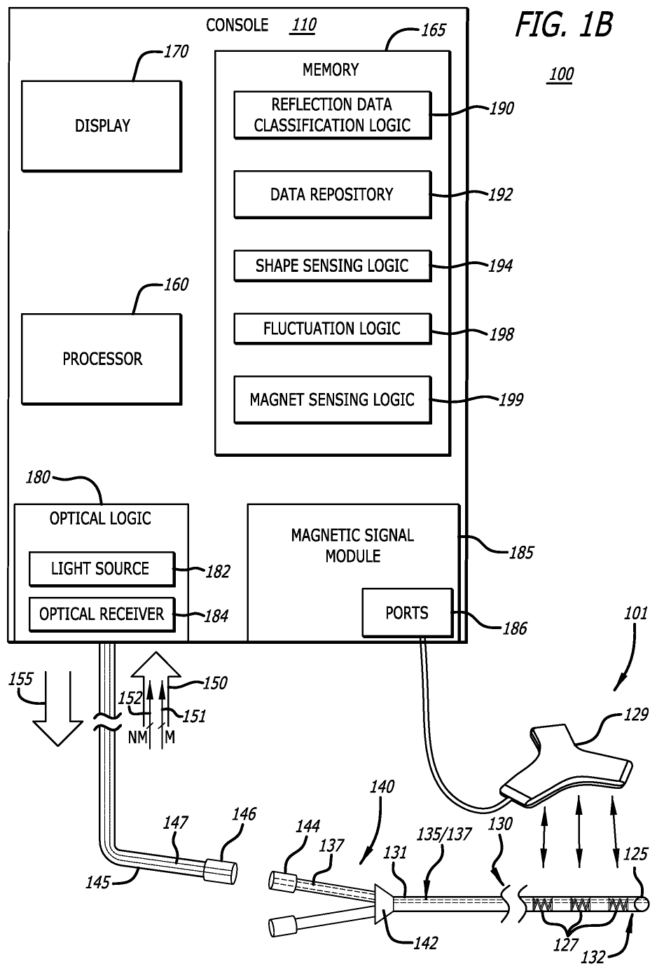

Referring to FIG. 1A, an illustrative embodiment of a medical instrument monitoring system including a medical instrument with optic shape sensing capabilities and a magnetic location system (MLS) 101 is shown in accordance with some embodiments. As shown, the system 100 generally includes a console 110, a stylet assembly 119 communicatively coupled to the console 110 and the magnetic field sensor 129 also coupled with the console 110. For this embodiment, the stylet assembly 119 includes an elongate probe (e.g., stylet) 120 on its distal end 122 and a console connector 133 on its proximal end 124. The console connector 133 enables the stylet assembly 119 to be operably connected to the console 110 via an interconnect 145 including one or more optical fibers 147 (hereinafter, “optical fiber(s)”) and a conductive medium terminated by a single optical/electric connector 146 (or terminated by dual connectors). Herein, the connector 146 is configured to engage (mate) with the console connector 133 to allow for the propagation of light between the console 110 and the stylet assembly 119 as well as the propagation of electrical signals from the stylet 120 to the console 110.

An exemplary implementation of the console 110 includes a processor 160, a memory 165, a display 170 and optical logic 180, although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Publication No. 2019/0237902, the entire contents of which are incorporated by reference herein. The processor 160, with access to the memory 165 (e.g., non-volatile memory or non-transitory, computer-readable medium), is included to control functionality of the console 110 during operation. As shown, the display 170 may be a liquid crystal diode (LCD) display integrated into the console 110 and employed as a user interface to display information to the clinician, especially during a catheter placement procedure (e.g., cardiac catheterization). In another embodiment, the display 170 may be separate from the console 110. Although not shown, a user interface is configured to provide user control of the console 110.

For both of these embodiments, the content depicted by the display 170 may change according to which mode the stylet 120 is configured to operate: optical, TLS, ECG, or another modality. In TLS mode, the content rendered by the display 170 may constitute a two-dimensional (2D) or three-dimensional (3D) representation of the physical state (e.g., length, shape, form, and/or orientation) of the stylet 120 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below

According to one embodiment of the disclosure, an activation control 126, included on the stylet assembly 119, may be used to set the stylet 120 into a desired operating mode and selectively alter operability of the display 170 by the clinician to assist in medical device placement. For example, based on the modality of the stylet 120, the display 170 of the console 110 can be employed for optical modality-based guidance during catheter advancement through the vasculature or TLS modality to determine the physical state (e.g., length, form, shape, orientation, etc.) of the stylet 120. In one embodiment, information from multiple modes, such as optical, TLS or ECG for example, may be displayed concurrently (e.g., at least partially overlapping in time).

Referring still to FIG. 1A, the optical logic 180 is configured to support operability of the stylet assembly 119 and enable the return of information to the console 110, which may be used to determine the physical state associated with the stylet 120 along with monitored electrical signals such as ECG signaling via an electrical signaling logic 181 that supports receipt and processing of the received electrical signals from the stylet 120, or more specifically from the ECG electrode 125, (e.g., ports, analog-to-digital conversion logic, etc.). The physical state of the stylet 120 may be based on changes in characteristics of the reflected light signals 150 received at the console 110 from the stylet 120. The characteristics may include shifts in wavelength caused by strain on certain regions of the core fibers integrated within an optical fiber core 135 positioned within or operating as the stylet 120, as shown below. As discussed herein, the optical fiber core 135 may be comprised of core fibers 137₁-137_{M} (M=1 for a single core, and M>2 for a multi-core), where the core fibers 137₁-137_{M} may collectively be referred to as core fiber(s) 137. Unless otherwise specified or the instant embodiment requires an alternative interpretation, embodiments discussed herein will refer to a multi-core optical fiber 135. From information associated with the reflected light signals 150, the console 110 may determine (through computation or extrapolation of the wavelength shifts) the physical state of the stylet 120, and also that of a catheter 195 configured to receive the stylet 120.

According to one embodiment of the disclosure, as shown in FIG. 1A, the optical logic 180 may include a light source 182 and an optical receiver 184. The light source 182 is configured to transmit the incident light 155 (e.g., broadband) for propagation over the optical fiber(s) 147 included in the interconnect 145, which are optically connected to the multi-core optical fiber core 135 within the stylet 120. In one embodiment, the light source 182 is a tunable swept laser, although other suitable light sources can also be employed in addition to a laser, including semi-coherent light sources, LED light sources, etc.

The optical receiver 184 is configured to: (i) receive returned optical signals, namely reflected light signals 150 received from optical fiber-based reflective gratings (sensors) fabricated within each core fiber of the multi-core optical fiber 135 deployed within the stylet 120, and (ii) translate the reflected light signals 150 into reflection data (from a data repository 192), namely data in the form of electrical signals representative of the reflected light signals including wavelength shifts caused by strain. The reflected light signals 150 associated with different spectral widths may include reflected light signals 151 provided from sensors positioned in the center core fiber (reference) of the multi-core optical fiber 135 and reflected light signals 152 provided from sensors positioned in the periphery core fibers of the multi-core optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the processor 160, which governs their operation. Also, the optical receiver 184 is operably coupled to provide the reflection data (from the data repository 192) to the memory 165 for storage and processing by reflection data classification logic 190. The reflection data classification logic 190 may be configured to: (i) identify which core fibers pertain to which of the received reflection data (from the data repository 192) and (ii) segregate the reflection data stored within the data repository 192 provided from reflected light signals 150 pertaining to similar regions of the stylet 120 or spectral widths into analysis groups. The reflection data for each analysis group is made available to shape sensing logic 194 for analytics.

According to one embodiment of the disclosure, the shape sensing logic 194 is configured to compare wavelength shifts measured by sensors deployed in each periphery core fiber at the same measurement region of the stylet 120 (or same spectral width) to the wavelength shift at a center core fiber of the multi-core optical fiber 135 positioned along central axis and operating as a neutral axis of bending. From these analytics, the shape sensing logic 194 may determine the shape the core fibers have taken in three-dimensional space and may further determine the current physical state of the catheter 195 in three-dimensional space for rendering on the display 170.

According to one embodiment of the disclosure, the shape sensing logic 194 may generate a rendering of the current physical state of the stylet 120 (and potentially the catheter 195), based on heuristics or run-time analytics. For example, the shape sensing logic 194 may be configured in accordance with machine-learning techniques to access the data repository 192 with pre-stored data (e.g., images, etc.) pertaining to different regions of the stylet 120 (or catheter 195) in which reflected light from core fibers have previously experienced similar or identical wavelength shifts. From the pre-stored data, the current physical state of the stylet 120 (or catheter 195) may be rendered. Alternatively, as another example, the shape sensing logic 194 may be configured to determine, during run-time, changes in the physical state of each region of the multi-core optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the multi-core optical fiber 135 to the wavelength shift generated by a sensor of the center core fiber at the same cross-sectional region. It is contemplated that other processes and procedures may be performed to utilize the wavelength shifts as measured by sensors along each of the core fibers within the multi-core optical fiber 135 to render appropriate changes in the physical state of the stylet 120 (and/or catheter 195), especially to enable guidance of the stylet 120, when positioned at a distal tip of the catheter 195, within the vasculature of the patient and at a desired destination within the body.

The console 110 may further include electrical signaling logic 181, which is positioned to receive one or more electrical signals from the stylet 120. The stylet 120 is configured to support both optical connectivity as well as electrical connectivity. The electrical signaling logic 181 receives the electrical signals (e.g., ECG signals) from the stylet 120 via the conductive medium. The electrical signals may be processed by electrical signal logic 196, executed by the processor 160, to determine ECG waveforms for display.

Additionally, the console 110 includes a fluctuation logic 198 that is configured to analyze at least a subset of the wavelength shifts measured by sensors deployed in each of the core fibers 137. In particular, the fluctuation logic 198 is configured to analyze wavelength shifts measured by sensors of core fibers 137, where such corresponds to an analysis of the fluctuation of the distal tip of the stylet 120 (or “tip fluctuation analysis”). In some embodiments, the fluctuation logic 198 measures analyzes the wavelength shifts measured by sensors at a distal end of the core fibers 137. The tip fluctuation analysis includes at least a correlation of detected movements of the distal tip of the stylet 120 (or other medical device or instrument) with experiential knowledge comprising previously detected movements (fluctuations), and optionally, other current measurements such as ECG signals. The experiential knowledge may include previously detected movements in various locations within the vasculature (e.g., SVC, Inferior Vena Cava (IVC), right atrium, azygos vein, other blood vessels such as arteries and veins) under normal, healthy conditions and in the presence of defects (e.g., vessel constriction, vasospasm, vessel occlusion, etc.). Thus, the tip fluctuation analysis may result in a confirmation of tip location and/or detection of a defect affecting a blood vessel.

It should be noted that the fluctuation logic 198 need not perform the same analyses as the shape sensing logic 194. For instance, the shape sensing logic 194 determines a three-dimensional shape of the stylet 120 by comparing wavelength shifts in outer core fibers of a multi-core optical fiber to a center, reference core fiber. The fluctuation logic 198 may instead correlate the wavelength shifts to previously measured wavelength shifts and optionally other current measurements without distinguishing between wavelength shifts of outer core fibers and a center, reference core fiber as the tip fluctuation analysis need not consider direction or shape within a three-dimensional space.

In some embodiments, e.g., those directed at tip location confirmation, the analysis of the fluctuation logic 198 may utilize electrical signals (e.g., ECG signals) measured by the electrical signaling logic 181. For example, the fluctuation logic 198 may compare the movements of a subsection of the stylet 120 (e.g., the distal tip) with electrical signals indicating impulses of the heart (e.g., the heartbeat). Such a comparison may reveal whether the distal tip is within the SVC or the right atrium based on how closely the movements correspond to a rhythmic heartbeat.

In various embodiments, a display message and/or alert may be generated based on the fluctuation analysis. For instance, the fluctuation logic 198 may generate a graphic illustrating the detected fluctuation compared to previously detected tip fluctuations and/or the anatomical movements of the patient body such as rhythmic pulses of the heart and/or expanding and contracting of the lungs. In one embodiment, such a graphic may include a dynamic visualization of the present medical device moving in accordance with the detected fluctuations adjacent to a secondary medical device moving in accordance with previously detected tip fluctuations. In some embodiments, the location of a subsection of the medical device may be obtained from the shape sensing logic 194 and the dynamic visualization may be location-specific (e.g., such that the previously detected fluctuations illustrate expected fluctuations for the current location of the subsection). In alternative embodiments, the dynamic visualization may illustrate a comparison of the dynamic movements of the subsection to one or more subsections moving in accordance with previously detected fluctuations of one or more defects affecting the blood vessel.

According to one embodiment of the disclosure, the fluctuation logic 198 may determine whether movements of one or more subsections of the stylet 120 indicate a location of a particular subsection of the stylet 120 or a defect affecting a blood vessel, based on heuristics or run-time analytics. For example, the fluctuation logic 198 may be configured in accordance with machine-learning techniques to access the data repository 192 with pre-stored data (e.g., experiential knowledge of previously detected tip fluctuation data, etc.) pertaining to different regions (subsections) of the stylet 120. Specifically, such an embodiment may include processing of a machine-learning model trained using the experiential knowledge, where the detected fluctuations serve as input to the trained model and processing of the trained model results in a determination as to how closely the detected fluctuations correlate to one or more locations within the vasculature of the patient and/or one or more defects affecting a blood vessel.

In some embodiments, the fluctuation logic 198 may be configured to determine, during run-time, whether movements of one or more subsections of the stylet 120 (and the catheter 130, see FIG. 1B) indicate a location of a particular subsection of the stylet 120 or a defect affecting a blood vessel, based on at least (i) resultant wavelength shifts experienced by the core fibers 137 within the one or more subsections, and (ii) the correlation of these wavelength shifts generated by sensors positioned along different core fibers at the same cross-sectional region of the stylet 120 (or the catheter 130) to previously detected wavelength shifts generated by corresponding sensors in a core fiber at the same cross-sectional region. It is contemplated that other processes and procedures may be performed to utilize the wavelength shifts as measured by sensors along each of the core fibers 137 to render appropriate movements in the distal tip of the stylet 120 and/or the catheter 130.

As stated above, the medical instrument monitoring system 100 further includes the magnetic location system (MLS) 101. The MLS 101 enables the clinician to quickly locate and confirm the position and/or orientation of segments of the stylet 120 (or catheter 195) during initial placement into and advancement through the vasculature of the patient. Specifically, the MLS 101 detects magnetic fields generated by a plurality of magnetic elements 127 coupled with the stylet 120. In one embodiment, each of the magnetic elements 127 can be tracked using the teachings of one or more of the following U.S. Pat. Nos.: 5,775,322; 5,879,297; 6,129,668; 6,216,028; and 6,263,230. The contents of the afore-mentioned U.S. patents are incorporated herein by reference in their entireties. The MLS 101 also detects an orientation of the magnetic elements 127, i.e., the direction in which each of the magnetic elements 127 is pointing, thus further assisting accurate placement of the stylet 120. The MLS 101 further assists the clinician in determining when a malposition of a tip 123 of the stylet 120 has occurred, such as in the case where the tip 123 has deviated from a desired venous path into another vein.

As mentioned above, the MLS 101 may utilize the stylet 120 to enable the catheter 195 to be tracked during its advancement through the vasculature. The magnetic elements 127 are disposed along the stylet 120. In some embodiments, one or more magnetic elements 127 may be disposed at the distal tip 123 of the stylet 120 and additional magnetic elements 127 may be dispersed along a length of the stylet 120 extending away from the distal tip 123. The stylet 120 may include 1, 2, 3, 4, 5, or more magnetic elements 127. The stylet 120 may also include an ECG electrode 125 at the distal tip 123.

Each magnet element 127 may be longitudinal in shape and each magnet element 127 may disposed parallel with (or tangent to) the stylet 120. In other words, each magnetic element 127 may be attached to the stylet 120 so that a longitudinal axis of the magnetic element 127 is aligned with a longitudinal axis of the stylet 120. Each magnet element 127 includes a north magnetic pole 128A and a south magnetic pole 128B located at opposite ends of the magnetic element 127. In some embodiments, each north magnetic pole 128A may point in a proximal direction and each south magnetic pole 128A may point in a distal direction or vice versa. In some embodiments, the magnetic elements 127 may be permanent magnets. In further embodiments, the magnetic elements 127 may include magnetized portions of a ferrous material. In other embodiments, the magnetic elements 127 may vary from the design in not only shape, but also composition, number, size, magnetic type, and position along the stylet 120.

For example, in one embodiment, the plurality of magnetic elements 127 may be replaced with an electromagnetic assembly, such as an electromagnetic coil, which produces a magnetic field for detection by the magnetic field sensor 129. Another example of an assembly usable here can be found in U.S. Pat. No. 5,099,845 entitled “Medical Instrument Location Means,” which is incorporated herein by reference in its entirety. Yet other examples of stylets including magnetic elements that can be employed with the MLS 101 can be found in U.S. application Ser. No. 11/466,602, filed Aug. 23, 2006, and entitled “Stylet Apparatuses and Methods of Manufacture,” published as U.S. Publication No. 2007/0049846, which is incorporated herein by reference in its entirety. These and other variations are therefore contemplated by embodiments of the disclosure.

The magnetic field sensor 129 is employed by the system 100 during instrument monitoring operation to detect magnetic fields defined by the magnetic elements 127 of the stylet 120. The medical instrument monitoring system 100 includes a magnetic field sensor 129 configured to sense a magnetic field defined by each magnetic element 127 which sensing includes determining a position of the magnetic element 127 in three-dimensional space with respect to the magnetic field sensor 129. The sensing further includes determining an orientation of the magnetic element 127, i.e., the direction of the north and south poles 128A, 128B. An exemplary system for coupling magnetic elements with a medical device, inserting the medical device within a human body, and determining the position and orientation the magnetic elements with the human body is shown and described in U.S. Pat. No. 8,388,541 entitled “Integrated System for Intravascular Placement of a Catheter,” the entire contents of which is incorporated by reference herein.

According to one embodiment of the disclosure, the magnet sensing logic 199 may generate a rendering of the positions and orientations of the magnetic elements 127. For example, a magnetic element 127 may define a magnetic field which is sensed by the magnetic field sensor 129. The magnetic sensing logic 199 receives magnetic field data from the magnetic field sensor 129 and therefrom determines a three-dimensional position of the magnetic element 127. As the magnetic field is defined by north and south poles 128A, 128B, magnetic sensing logic 199 also determines the orientation of the magnetic element 127. As each magnetic element 127 is coupled with an attachment segment of the stylet 120, the magnet sensing logic 199 determines a three-dimensional position of the attachment segment of the stylet 120. Further, as each longitudinal axis of the magnetic element 127 is alignment with a longitudinal axis of the attachment segment of the stylet 120, the magnet sensing logic 199 determines a three-dimensional direction/orientation of the attachment segment. By way of summary, the shape sensing logic 194 determines the three-dimensional shape of the stylet 120 and the magnet sensing logic 199 determines the three-dimensional position and orientation of the attachment segments of the stylet 120. Hence, the shape sensing logic 194 in combination with the magnet sensing logic 199, determines the three-dimensional shape, position, and orientation of the stylet 120.

According to one embodiment of the disclosure, the magnet sensing logic 199 may generate a rendering of the current position and orientation of the catheter 130 based on heuristics or run-time analytics. For example, the magnet sensing logic 199 may be configured in accordance with machine-learning (ML) techniques to access the data repository 192 with pre-stored data (e.g., representation of images, etc.) pertaining to different magnetic element positions and orientations with respect to the magnetic field sensor 129 in which the magnetic field sensor 129 sensed similar or identical magnetic fields. From the pre-stored data, the current position and orientation of the stylet 120 may be rendered. For example, a machine-learning model may be trained to generate scores of images of the 3D images of the catheter 130 at various positionings and orientations, where the score represents a probability that a particular 3D image corresponds to received positioning and orientation data and where the ML model is trained using the pre-stored data. The ML model may be configured to receive positioning and orientation data (e.g., wavelength shifts, a three-dimensional position as discussed above, etc.) and provide a score for one or more 3D images.

Further the magnet sensing logic 199 may generate a rendering of the current position and orientation of a tip 123 of the stylet 120 based on heuristics or run-time analytics. For example, the magnet sensing logic 199 may be configured in accordance with machine-learning techniques to access the data repository 192 with pre-stored data (e.g., images, etc.) pertaining to different magnetic element positions and orientations with respect to the magnetic field sensor 129 in combination an ECG signal. From the pre-stored data, the current position and orientation of the catheter 130 may be rendered.

Referring to FIG. 1B, an alternative exemplary embodiment of a medical instrument monitoring system 100 is shown. Herein, the medical instrument monitoring system 100 features a console 110 and a medical instrument corresponding to a catheter 130 for this embodiment is communicatively coupled to the console 110. The catheter 130 features an integrated tubing with two or more lumen extending between a proximal end 131 and a distal end 132 of the integrated tubing. The integrated tubing (sometimes referred to as “catheter tubing”) is in communication with one or more extension legs 140 via a bifurcation hub 142. An optical-based catheter connector 144 may be included on a proximal end of at least one of the extension legs 140 to enable the catheter 130 to operably connect to the console 110 via an interconnect 145 or another suitable component. Herein, the interconnect 145 may include a connector 146 that, when coupled to the optical-based catheter connector 144, establishes optical connectivity between one or more optical fibers 147 (hereinafter, “optical fiber(s)”) included as part of the interconnect 145 and core fibers 137 deployed within the catheter 130 and integrated into the tubing. Alternatively, a different combination of connectors, including one or more adapters, may be used to optically connect the optical fiber(s) 147 to the core fibers 137 within the catheter 130. The core fibers 137 deployed within the catheter 130 as illustrated in FIG. 1B include the same characteristics and perform the same functionalities as the core fibers 137 deployed within the stylet 120 of FIG. 1A. Similar to the stylet 120 of FIG. 1A, the catheter 130 includes a plurality of magnetic elements 127 disposed along a length of the catheter 130 and an ECG electrode 125 disposed at the distal end 132.

The optical logic 180 is configured to support graphical rendering of the catheter 130, most notably the integrated tubing of the catheter 130, based on characteristics of the reflected light signals 150 received from the catheter 130. The characteristics may include shifts in wavelength caused by strain on certain regions of the core fibers 137 integrated within (or along) a wall of the integrated tubing, which may be used to determine (through computation or extrapolation of the wavelength shifts) the physical state of the catheter 130, notably its integrated tubing or a portion of the integrated tubing such as a tip or distal end of the tubing to read fluctuations (real-time movement) of the tip (or distal end).

More specifically, the optical logic 180 includes a light source 182. The light source 182 is configured to transmit the broadband incident light 155 for propagation over the optical fiber(s) 147 included in the interconnect 145, which are optically connected to multiple core fibers 137 within the catheter tubing. Herein, the optical receiver 184 is configured to: (i) receive returned optical signals, namely reflected light signals 150 received from optical fiber-based reflective gratings (sensors) fabricated within each of the core fibers 137 deployed within the catheter 130, and (ii) translate the reflected light signals 150 into reflection data (from data repository 192), namely data in the form of electrical signals representative of the reflected light signals including wavelength shifts caused by strain. The reflected light signals 150 associated with different spectral widths include reflected light signals 151 provided from sensors positioned in the center core fiber (reference) of the catheter 130 and reflected light signals 152 provided from sensors positioned in the outer core fibers of the catheter 130, as described below.

As noted above, the shape sensing logic 194 is configured to compare wavelength shifts measured by sensors deployed in each outer core fiber at the same measurement region of the catheter (or same spectral width) to the wavelength shift at the center core fiber positioned along central axis and operating as a neutral axis of bending. From these analytics, the shape sensing logic 190 may determine the shape the core fibers have taken in 3D space and may further determine the current physical state of the catheter 130 in 3D space for rendering on the display 170.

According to one embodiment of the disclosure, the shape sensing logic 194 may generate a rendering of the current physical state of the catheter 130, especially the integrated tubing, based on heuristics or run-time analytics. For example, the shape sensing logic 194 may be configured in accordance with machine-learning techniques to access the data repository 192 with pre-stored data (e.g., images, etc.) pertaining to different regions of the catheter 130 in which the core fibers 137 experienced similar or identical wavelength shifts. From the pre-stored data, the current physical state of the catheter 130 may be rendered. Alternatively, as another example, the shape sensing logic 194 may be configured to determine, during run-time, changes in the physical state of each region of the catheter 130, notably the tubing, based on at least (i) resultant wavelength shifts experienced by the core fibers 137 and (ii) the relationship of these wavelength shifts generated by sensors positioned along different outer core fibers at the same cross-sectional region of the catheter 130 to the wavelength shift generated by a sensor of the center core fiber at the same cross-sectional region. It is contemplated that other processes and procedures may be performed to utilize the wavelength shifts as measured by sensors along each of the core fibers 137 to render appropriate changes in the physical state of the catheter 130.

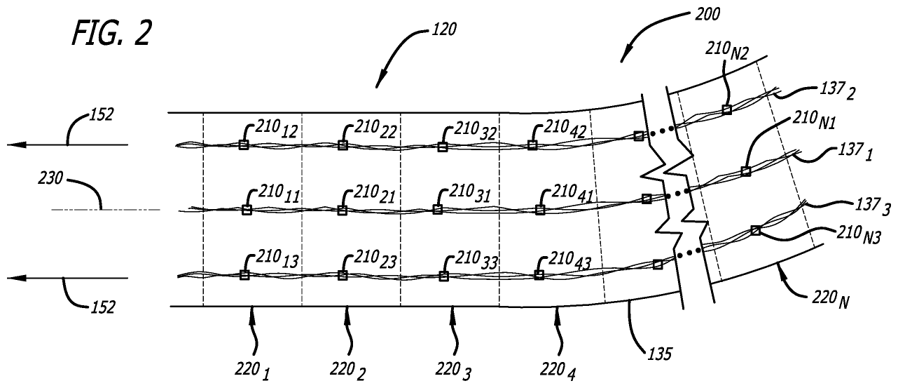

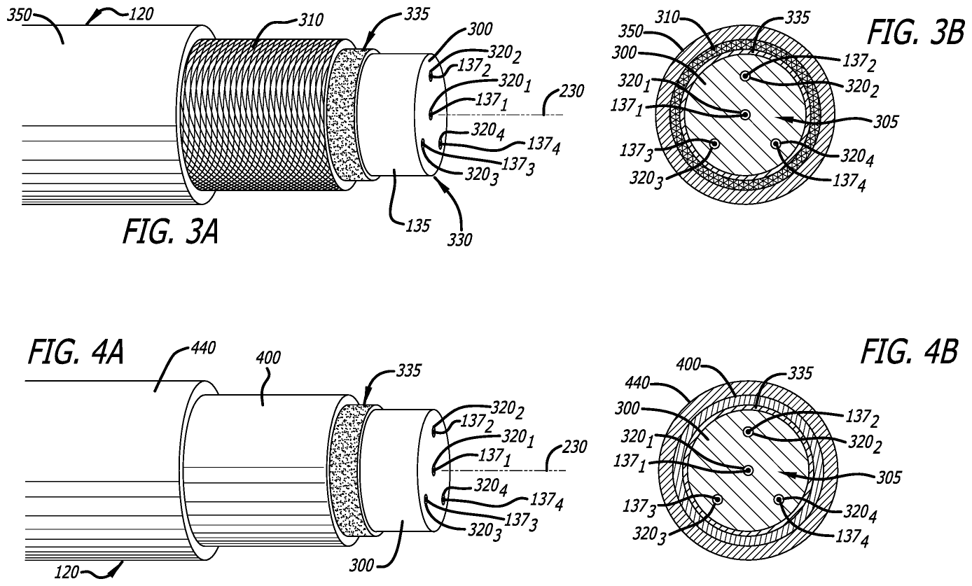

Referring to FIG. 2, an exemplary embodiment of a structure of a section of the multi-core optical fiber included within the stylet 120 of FIG. 1A is shown in accordance with some embodiments. The multi-core optical fiber section 200 of the multi-core optical fiber 135 depicts certain core fibers 137₁-137_{M} (M≥2, M=4 as shown, see FIG. 3A) along with the spatial relationship between sensors (e.g., reflective gratings) 210₁₁-210 _{NM} (N≥2; M≥2) present within the core fibers 137₁-137_{M}, respectively. As noted above, the core fibers 137₁-137_{M} may be collectively referred to as “the core fibers 137.”

As shown, the section 200 is subdivided into a plurality of cross-sectional regions 220₁-220_{N}, where each cross-sectional region 220₁-220_{N} corresponds to reflective gratings 210₁₁-210₁₄ . . . 210_{N1}-210_{N4}. Some or all of the cross-sectional regions 220₁ . . . 220_{N} may be static (e.g., prescribed length) or may be dynamic (e.g., vary in size among the regions 220₁ . . . 220_{N}). A first core fiber 137₁ is positioned substantially along a center (neutral) axis 230 while core fiber 137₂ may be oriented within the cladding of the multi-core optical fiber 135, from a cross-sectional, front-facing perspective, to be position on “top” the first core fiber 137₁. In this deployment, the core fibers 137₃ and 137₄ may be positioned “bottom left” and “bottom right” of the first core fiber 137₁. As examples, FIGS. 3A-4B provides illustrations of such.

Referencing the first core fiber 137₁ as an illustrative example, when the stylet 120 is operative, each of the reflective gratings 210₁-210_{N} reflects light for a different spectral width. As shown, each of the gratings 210₁-210 _{Ni} (1<i<M) is associated with a different, specific spectral width, which would be represented by different center frequencies of f₁ . . . f_{N}, where neighboring spectral widths reflected by neighboring gratings are non-overlapping according to one embodiment of the disclosure.

Herein, positioned in different core fibers 137₂-137₃ but along at the same cross-sectional regions 220-220_{N} of the multi-core optical fiber 135, the gratings 210₁₂-210_{N2} and 210₁₃-210_{N3} are configured to reflect incoming light at same (or substantially similar) center frequency. As a result, the reflected light returns information that allows for a determination of the physical state of the optical fibers 137 (and the stylet 120) based on wavelength shifts measured from the returned, reflected light. In particular, strain (e.g., compression or tension) applied to the multi-core optical fiber 135 (e.g., at least core fibers 137₂-137₃) results in wavelength shifts associated with the returned, reflected light. Based on different locations, the core fibers 137₁-137₄ experience different types and degree of strain based on angular path changes as the stylet 120 advances in the patient.

For example, with respect to the multi-core optical fiber section 200 of FIG. 2, in response to angular (e.g., radial) movement of the stylet 120 is in the left-veering direction, the fourth core fiber 137₄ (see FIG. 3A) of the multi-core optical fiber 135 with the shortest radius during movement (e.g., core fiber closest to a direction of angular change) would exhibit compression (e.g., forces to shorten length). At the same time, the third core fiber 137₃ with the longest radius during movement (e.g., core fiber furthest from the direction of angular change) would exhibit tension (e.g., forces to increase length). As these forces are different and unequal, the reflected light from reflective gratings 210_{N2} and 210_{N3} associated with the core fiber 137₂ and 137₃ will exhibit different changes in wavelength. The differences in wavelength shift of the reflected light signals 150 can be used to extrapolate the physical configuration of the stylet 120 by determining the degrees of wavelength change caused by compression/tension for each of the periphery fibers (e.g., the second core fiber 137₂ and the third core fiber 137₃) in comparison to the wavelength of the reference core fiber (e.g., first core fiber 137₁) located along the neutral axis 230 of the multi-core optical fiber 135. These degrees of wavelength change may be used to extrapolate the physical state of the stylet 120. The reflected light signals 150 are reflected back to the console 110 via individual paths over a particular core fiber 137₁-137_{M}.

Referring to FIG. 3A, a first exemplary embodiment of the stylet of FIG. 1A supporting both an optical and electrical signaling is shown in accordance with some embodiments. Herein, the stylet 120 features a centrally located multi-core optical fiber 135, which includes a cladding 300 and a plurality of core fibers 137₁-137_{M} (M>2; M=4) residing within a corresponding plurality of lumens 320₁-320_{M}. While the multi-core optical fiber 135 is illustrated within four (4) core fibers 137₁-137₄, a greater number of core fibers 137₁-137_{M} (M>4) may be deployed to provide a more detailed three-dimensional sensing of the physical state (e.g., shape, etc.) of the multi-core optical fiber 135 and the stylet 120 deploying the optical fiber 135.

For this embodiment of the disclosure, the multi-core optical fiber 135 is encapsulated within a concentric braided tubing 310 positioned over a low coefficient of friction layer 335. The braided tubing 310 may feature a “mesh” construction, in which the spacing between the intersecting conductive elements is selected based on the degree of rigidity desired for the stylet 120, as a greater spacing may provide a lesser rigidity, and thereby, a more pliable stylet 120.

According to this embodiment of the disclosure, as shown in FIGS. 3A-3B, the core fibers 137₁-137₄ include (i) a central core fiber 137₁ and (ii) a plurality of periphery core fibers 137₂-137₄, which are maintained within lumens 320₁-320₄ formed in the cladding 300. According to one embodiment of the disclosure, one or more of the lumen 320₁-320₄ may be configured with a diameter sized to be greater than the diameter of the core fibers 137₁-137₄. By avoiding a majority of the surface area of the core fibers 137₁-137₄ from being in direct physical contact with a wall surface of the lumens 3201-3204, the wavelength changes to the incident light are caused by angular deviations in the multi-core optical fiber 135 thereby reducing influence of compression and tension forces being applied to the walls of the lumens 320₁-320_{M}, not the core fibers 137₁-137_{M} themselves.

As further shown in FIGS. 3A-3B, the core fibers 137₁-137₄ may include central core fiber 137₁ residing within a first lumen 320₁ formed along the first neutral axis 230 and a plurality of core fibers 137₂-137₄ residing within lumens 320₂-320₄ each formed within different areas of the cladding 300 radiating from the first neutral axis 230. In general, the core fibers 137₂-137₄, exclusive of the central core fiber 137₁, may be positioned at different areas within a cross-sectional area 305 of the cladding 300 to provide sufficient separation to enable three-dimensional sensing of the multi-core optical fiber 135 based on changes in wavelength of incident light propagating through the core fibers 137₂-137₄ and reflected back to the console for analysis.

For example, where the cladding 300 features a circular cross-sectional area 305 as shown in FIG. 3B, the core fibers 137₂-137₄ may be positioned substantially equidistant from each other as measured along a perimeter of the cladding 300, such as at “top” (12 o'clock), “bottom-left” (8 o'clock) and “bottom-right” (4 o'clock) locations as shown. Hence, in general terms, the core fibers 137₂-137₄ may be positioned within different segments of the cross-sectional area 305. Where the cross-sectional area 305 of the cladding 300 has a distal tip 330 and features a polygon cross-sectional shape (e.g., triangular, square, rectangular, pentagon, hexagon, octagon, etc.), the central core fiber 137₁ may be located at or near a center of the polygon shape, while the remaining core fibers 137₂-137_{M} may be located proximate to angles between intersecting sides of the polygon shape.

Referring still to FIGS. 3A-3B, operating as the conductive medium for the stylet 120, the braided tubing 310 provides mechanical integrity to the multi-core optical fiber 135 and operates as a conductive pathway for electrical signals. For example, the braided tubing 310 may be exposed to a distal tip of the stylet 120. The cladding 300 and the braided tubing 310, which is positioned concentrically surrounding a circumference of the cladding 300, are contained within the same insulating layer 350. The insulating layer 350 may be a sheath or conduit made of protective, insulating (e.g., non-conductive) material that encapsulates both for the cladding 300 and the braided tubing 310, as shown.

Referring to FIG. 4A, a second exemplary embodiment of the stylet of FIG. 1A is shown in accordance with some embodiments. Referring now to FIG. 4A, a second exemplary embodiment of the stylet 120 of FIG. 1A supporting both an optical and electrical signaling is shown. Herein, the stylet 120 features the multi-core optical fiber 135 described above and shown in FIG. 3A, which includes the cladding 300 and the first plurality of core fibers 137₁-137_{M} (M≥3; M=4 for embodiment) residing within the corresponding plurality of lumens 320₁-320_{M}. For this embodiment of the disclosure, the multi-core optical fiber 135 includes the central core fiber 137₁ residing within the first lumen 320₁ formed along the first neutral axis 230 and the second plurality of core fibers 137₂-137₄ residing within corresponding lumens 320₂-320₄ positioned in different segments within the cross-sectional area 305 of the cladding 300. Herein, the multi-core optical fiber 135 is encapsulated within a conductive tubing 400. The conductive tubing 400 may feature a “hollow” conductive cylindrical member concentrically encapsulating the multi-core optical fiber 135.

Referring to FIGS. 4A-4B, operating as a conductive medium for the stylet 120 in the transfer of electrical signals (e.g., ECG signals) to the console, the conductive tubing 400 may be exposed up to a tip 410 of the stylet 120. For this embodiment of the disclosure, a conductive epoxy 420 (e.g., metal-based epoxy such as a silver epoxy) may be affixed to the tip 410 and similarly joined with a termination/connection point created at a proximal end 430 of the stylet 120. The cladding 300 and the conductive tubing 400, which is positioned concentrically surrounding a circumference of the cladding 300, are contained within the same insulating layer 440. The insulating layer 440 may be a protective conduit encapsulating both for the cladding 300 and the conductive tubing 400, as shown.

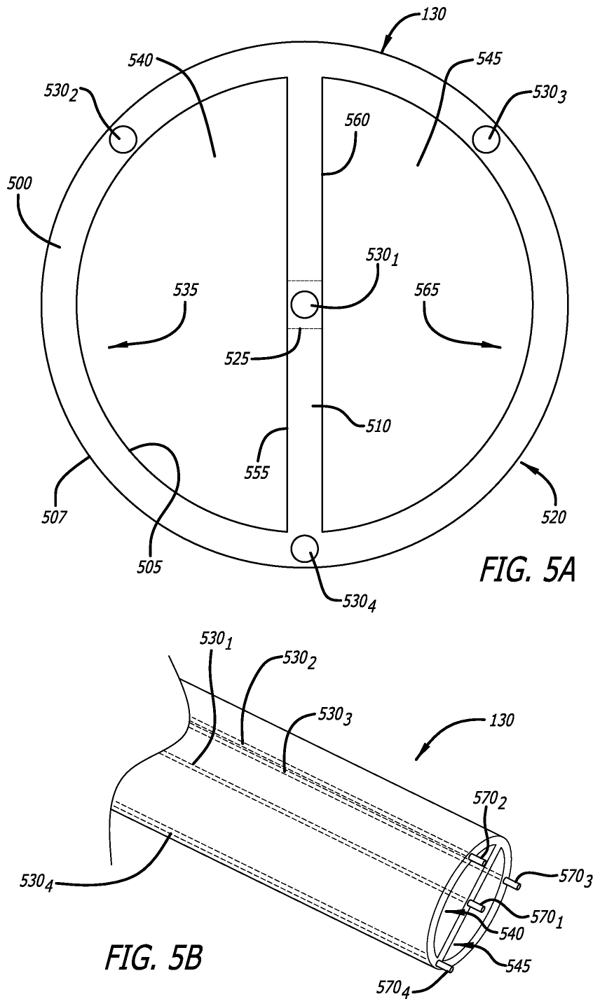

Referring to FIG. 5A, an elevation view of a first illustrative embodiment of a catheter including integrated tubing, a diametrically disposed septum, and micro-lumens formed within the tubing and septum is shown in accordance with some embodiments. Herein, the catheter 130 includes integrated tubing, the diametrically disposed septum 510, and the plurality of micro-lumens 530₁-530₄ which, for this embodiment, are fabricated to reside within the wall 500 of the integrated tubing of the catheter 130 and within the septum 510. In particular, the septum 510 separates a single lumen, formed by the inner surface 505 of the wall 500 of the catheter 130, into multiple lumen, namely two lumens 540 and 545 as shown. Herein, the first lumen 540 is formed between a first arc-shaped portion 535 of the inner surface 505 of the wall 500 forming the catheter 130 and a first outer surface 555 of the septum 510 extending longitudinally within the catheter 130. The second lumen 545 is formed between a second arc-shaped portion 565 of the inner surface 505 of the wall 500 forming the catheter 130 and a second outer surfaces 560 of the septum 510.

According to one embodiment of the disclosure, the two lumens 540 and 545 have approximately the same volume. However, the septum 510 need not separate the tubing into two equal lumens. For example, instead of the septum 510 extending vertically (12 o'clock to 6 o'clock) from a front-facing, cross-sectional perspective of the tubing, the septum 510 could extend horizontally (3 o'clock to 9 o'clock), diagonally (1 o'clock to 7 o'clock; 10 o'clock to 4 o'clock) or angularly (2 o'clock to 10 o'clock). In the later configuration, each of the lumens 540 and 545 of the catheter 130 would have a different volume.

With respect to the plurality of micro-lumens 530₁-530₄, the first micro-lumen 530₁ is fabricated within the septum 510 at or near the cross-sectional center 525 of the integrated tubing. For this embodiment, three micro-lumens 530₂-530₄ are fabricated to reside within the wall 500 of the catheter 130. In particular, a second micro-lumen 530₂ is fabricated within the wall 500 of the catheter 130, namely between the inner surface 505 and outer surface 507 of the first arc-shaped portion 535 of the wall 500. Similarly, the third micro-lumen 530₃ is also fabricated within the wall 500 of the catheter 130, namely between the inner and outer surfaces 505/507 of the second arc-shaped portion 555 of the wall 500. The fourth micro-lumen 530₄ is also fabricated within the inner and outer surfaces 505/507 of the wall 500 that are aligned with the septum 510.

According to one embodiment of the disclosure, as shown in FIG. 5A, the micro-lumens 530₂-530₄ are positioned in accordance with a “top-left” (10 o'clock), “top-right” (2 o'clock) and “bottom” (6 o'clock) layout from a front-facing, cross-sectional perspective. Of course, the micro-lumens 530₂-530₄ may be positioned differently, provided that the micro-lumens 530₂-530₄ are spatially separated along the circumference 520 of the catheter 130 to ensure a more robust collection of reflected light signals from the outer core fibers 570₂-570₄ when installed. For example, two or more of micro-lumens (e.g., micro-lumens 530₂ and 530₄) may be positioned at different quadrants along the circumference 520 of the catheter wall 500.

Referring to FIG. 5B, a perspective view of the first illustrative embodiment of the catheter of FIG. 5A including core fibers installed within the micro-lumens is shown in accordance with some embodiments. According to one embodiment of the disclosure, the second plurality of micro-lumens 530₂-530₄ are sized to retain corresponding outer core fibers 570₂-570₄, where the diameter of each of the second plurality of micro-lumens 530₂-530₄ may be sized just larger than the diameters of the outer core fibers 570₂-570₄. The size differences between a diameter of a single core fiber and a diameter of any of the micro-lumen 530₁-530₄ may range between 0.001 micrometers (μm) and 1000 μm, for example. As a result, the cross-sectional areas of the outer core fibers 570₂-570₄ would be less than the cross-sectional areas of the corresponding micro-lumens 530₂-530₄. A “larger” micro-lumen (e.g., micro-lumen 530₂) may better isolate external strain being applied to the outer core fiber 570₂ from strain directly applied to the catheter 130 itself. Similarly, the first micro-lumen 530₁ may be sized to retain the center core fiber 570₁, where the diameter of the first micro-lumen 530₁ may be sized just larger than the diameter of the center core fiber 570₁.

As an alternative embodiment of the disclosure, one or more of the micro-lumens 530₁-530₄ may be sized with a diameter that exceeds the diameter of the corresponding one or more core fibers 570₁-570₄. However, at least one of the micro-lumens 530₁-530₄ is sized to fixedly retain their corresponding core fiber (e.g., core fiber retained with no spacing between its lateral surface and the interior wall surface of its corresponding micro-lumen). As yet another alternative embodiment of the disclosure, all the micro-lumens 530₁-530₄ are sized with a diameter to fixedly retain the core fibers 570₁-570₄.

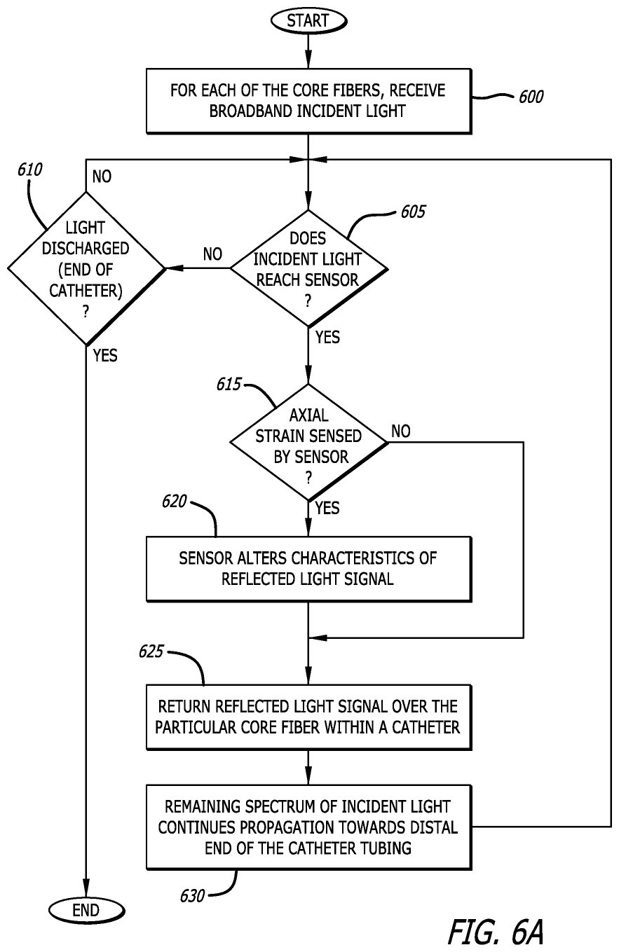

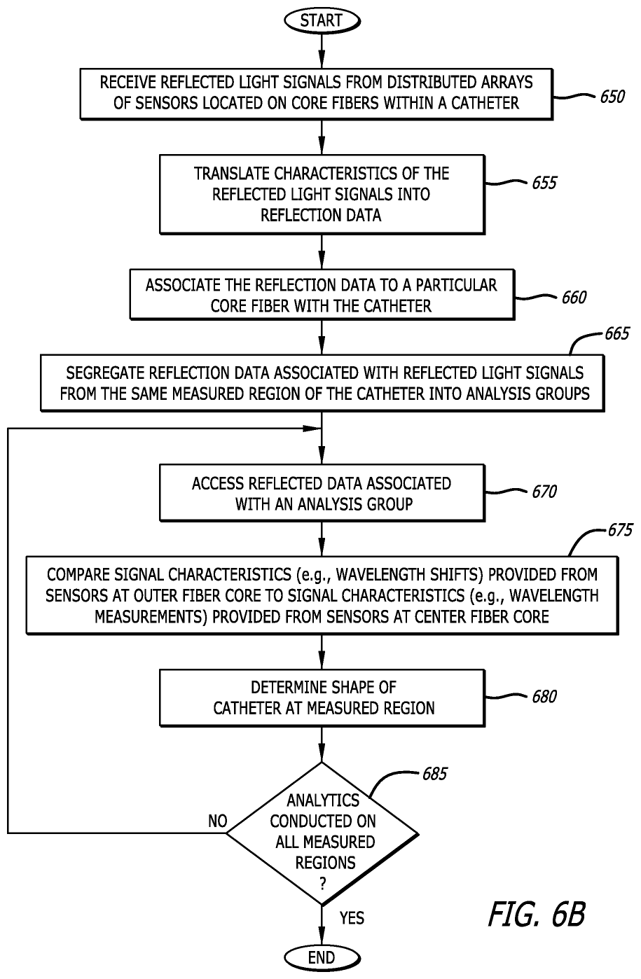

Referring to FIGS. 6A-6B, flowcharts of methods of operations conducted by the medical instrument monitoring system of FIGS. 1A-1B to achieve optic 3D shape sensing are shown in accordance with some embodiments. Herein, the catheter includes at least one septum spanning across a diameter of the tubing wall and continuing longitudinally to subdivide the tubing wall. The medial portion of the septum is fabricated with a first micro-lumen, where the first micro-lumen is coaxial with the central axis of the catheter tubing. The first micro-lumen is configured to retain a center core fiber. Two or more micro-lumen, other than the first micro-lumen, are positioned at different locations circumferentially spaced along the wall of the catheter tubing. For example, two or more of the second plurality of micro-lumens may be positioned at different quadrants along the circumference of the catheter wall.

Furthermore, each core fiber includes a plurality of sensors spatially distributed along its length between at least the proximal and distal ends of the catheter tubing. This array of sensors is distributed to position sensors at different regions of the core fiber to enable distributed measurements of strain throughout the entire length or a selected portion of the catheter tubing. These distributed measurements may be conveyed through reflected light of different spectral widths (e.g., specific wavelength or specific wavelength ranges) that undergoes certain wavelength shifts based on the type and degree of strain.

According to one embodiment of the disclosure, as shown in FIG. 6A, for each core fiber, broadband incident light is supplied to propagate through a particular core fiber (block 600). Unless discharged, upon the incident light reaching a sensor of a distributed array of sensors measuring strain on a particular core fiber, light of a prescribed spectral width associated with the first sensor is to be reflected back to an optical receiver within a console (blocks 605-610). Herein, the sensor alters characteristics of the reflected light signal to identify the type and degree of strain on the particular core fiber as measured by the first sensor (blocks 615-620). According to one embodiment of the disclosure, the alteration in characteristics of the reflected light signal may signify a change (shift) in the wavelength of the reflected light signal from the wavelength of the incident light signal associated with the prescribed spectral width. The sensor returns the reflected light signal over the core fiber and the remaining spectrum of the incident light continues propagation through the core fiber toward a distal end of the catheter tubing (blocks 625-630). The remaining spectrum of the incident light may encounter other sensors of the distributed array of sensors, where each of these sensors would operate as set forth in blocks 605-630 until the last sensor of the distributed array of sensors returns the reflected light signal associated with its assigned spectral width and the remaining spectrum is discharged as illumination.

Referring now to FIG. 6B, during operation, multiple reflected light signals are returned to the console from each of the plurality of core fibers residing within the corresponding plurality of micro-lumens formed within a catheter, such as the catheter of FIG. 1B. In particular, the optical receiver receives reflected light signals from the distributed arrays of sensors located on the center core fiber and the outer core fibers and translates the reflected light signals into reflection data, namely electrical signals representative of the reflected light signals including wavelength shifts caused by strain (blocks 650-655). The reflection data classification logic is configured to identify which core fibers pertain to which reflection data and segregate reflection data provided from reflected light signals pertaining to a particular measurement region (or similar spectral width) into analysis groups (block 660-665).

Each analysis group of reflection data is provided to shape sensing logic for analytics (block 670). Herein, the shape sensing logic compares wavelength shifts at each outer core fiber with the wavelength shift at the center core fiber positioned along central axis and operating as a neutral axis of bending (block 675). From this analytics, on all analytic groups (e.g., reflected light signals from sensors in all or most of the core fibers), the shape sensing logic may determine the shape the core fibers have taken in three-dimensional space, from which the shape sensing logic can determine the current physical state of the catheter in three-dimension space (blocks 680-685).

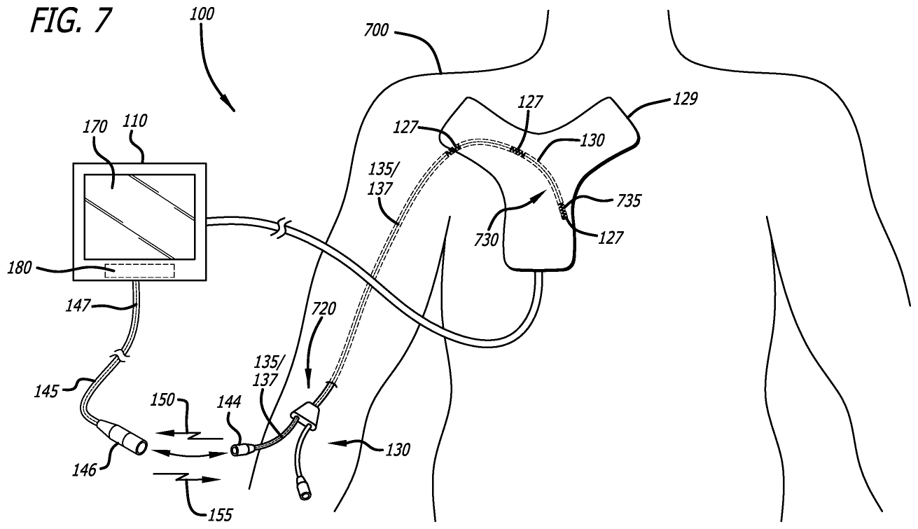

Referring to FIG. 7, an exemplary embodiment of the medical instrument monitoring system of FIG. 1B during operation and insertion of the catheter into a patient are shown in accordance with some embodiments. Herein, the catheter 130 generally includes the integrated tubing of the catheter 130 with a proximal portion 720 that generally remains exterior to the patient 700 and a distal portion 730 that generally resides within the patient vasculature after placement is complete. The (integrated) catheter tubing of the catheter 130 may be advanced to a desired position within the patient vasculature such as a distal end (or tip) 735 of the catheter tubing of the catheter 130 is proximate the patient's heart, such as in the lower one-third (⅓) portion of the Superior Vena Cava (“SVC”) for example. In some embodiments, various instruments may be disposed at the distal end 735 of the catheter 130 to measure pressure of blood in a certain heart chamber and in the blood vessels, view an interior of blood vessels, or the like. In alternative embodiments, such as those that utilize the stylet assembly of FIG. 1A and the catheter 195, such instruments may be disposed at a distal end of the stylet 120.

As seen in FIG. 7, the magnetic field sensor 129 is placed on the chest of the patient 700 during insertion of the catheter 130. The magnetic field sensor 129 is placed on the chest of the patient 700 in a predetermined location, such as through the use of external body landmarks, to enable the magnetic fields of the magnetic elements 127, disposed along the catheter 130 as described above, to be detected during transit of the catheter 130 through the patient vasculature. Again, as the magnetic elements 127 are coupled with the catheter 130, detection by the magnetic field sensor 129 of the magnetic fields defined by the magnetic elements 127 provides information to the clinician as to the position and orientation of the catheter 130. As one of the magnetic elements 127 may be co-terminal with the distal end 735 of the catheter 130, detection by the magnetic field sensor 129 of the magnetic field defined by the magnetic element 127 located at the distal end 735 provides information to the clinician as to the position and orientation of the distal end 735 of the catheter 130

In greater detail, the magnetic field sensor 129 is operably connected to the console 110 of the system 100. The console 110 includes a magnetic signal module 185 including connection ports 186, as shown in FIGS. 1A and 1B. The magnetic signal module 185 receives magnetic signals via the ports 186 from the magnetic field sensor 129 and converts the magnetic signals into magnetic data for processing by the magnetic sensing logic 199. In some embodiments, the magnetic signal module 185 may be located separate from the console 110. In such embodiments, the magnetic signal module 185 may include a transmitter for delivering magnetic data to the console 110.

Note that other connection schemes between the magnetic field sensor 129 and the system console 110 can also be used without limitation. As just described, the magnetic elements 127 are employed in the catheter 130 to enable the position of the catheter 130 to be observable relative to the magnetic field sensor 129 placed on the patient's chest. Detection by the magnetic field sensor 129 of the magnetic elements 127 is graphically displayed on the display 170 of the console 110 during operation. In this way, a clinician placing the catheter 130 is able to generally determine the location of the catheter 130 within the patient vasculature relative to the magnetic field sensor 129 and detect when catheter malposition, such as advancement of the catheter along an undesired vein, is occurring.

During advancement through a patient vasculature, the catheter 130 receives broadband incident light 155 from the console 110 via optical fiber(s) 147 within the interconnect 145, where the incident light 155 propagates along the core fibers 137 of the multi-core optical fiber 135 within the catheter tubing of the catheter 130. According to one embodiment of the disclosure, the connector 146 of the interconnect 145 terminating the optical fiber(s) 147 may be coupled to the optical-based catheter connector 144, which may be configured to terminate the core fibers 137 deployed within the catheter 130. Such coupling optically connects the core fibers 137 of the catheter 130 with the optical fiber(s) 147 within the interconnect 145. The optical connectivity is needed to propagate the incident light 155 to the core fibers 137 and return the reflected light signals 150 to the optical logic 180 within the console 110 over the interconnect 145. As described below in detail, the physical state of the catheter 130 may be ascertained based on analytics of the wavelength shifts of the reflected light signals 150.

Further during advancement through the patient vasculature, as the magnetic elements 127 are attached and aligned with the catheter 130, the magnetic field sensor 129 determines the three-dimensional position and orientation of the catheter 130, or more specifically the position and orientation of the magnetic elements 127 coupled with the catheter 130, with respect to the magnetic field sensor 129. As the magnetic field sensor 129 is applied to the patient at a defined location on the patient (e.g., at a location with respect to patient's heart), the three-dimensional position and orientation of the catheter 130 within the patient 700 may be ascertained by sensing the magnetic fields defined by the magnetic elements 127. By way of summary, the shape sensing functionality of the system 100 defines a physical state of the catheter 130, and the MLS 101 determines the location and orientation of the catheter 130 within the patient 700 with respect to the magnet field sensor 129.

According to one embodiment of the disclosure, the magnet sensing logic 199 may determine whether positions and/or orientations of one or more magnetic elements 127 of the stylet 120 (or the catheter 130) indicate a location of the stylet 120 based on heuristics or run-time analytics. For example, the magnet sensing logic 199 may be configured in accordance with machine-learning techniques to access the data repository 192 with pre-stored data (e.g., experiential knowledge of previously determined magnetic element locations) pertaining to different locations along the vasculature. Specifically, such an embodiment may include processing of a machine-learning model trained using the experiential knowledge, where the determined magnetic element locations serve as input to the trained model and processing of the trained model results in a determination as to how closely the determined the magnetic element locations correlate to one or more locations within the vasculature of the patient.

According to another embodiment of the disclosure, the magnet sensing logic 199 may determine whether positions and/or orientations of one or more magnetic elements 127 in combination with wavelength shifts of reflected light from the core fibers have previously indicated a location of the stylet 120 based on heuristics or run-time analytics. For example, the magnet sensing logic 199 may be configured in accordance with machine-learning techniques to access the data repository 192 with pre-stored data (e.g., experiential knowledge of previously determined combinations of magnetic element locations with wavelength shifts) pertaining to different locations along the vasculature. Specifically, such an embodiment may include processing of a machine-learning model trained using the experiential knowledge, where the determined magnetic element locations serve as input to the trained model and processing of the trained model results in a determination as to how closely the determined combinations of magnetic element locations with wavelength shifts correlate to one or more locations within the vasculature of the patient.

According to still a further embodiment of the disclosure, the magnet sensing logic 199 may determine whether a position and/or orientation of a magnetic element 127 disposed adjacent the tip 123 of the stylet 120 in combination an ECG signal have previously indicated a location of the tip 123 based on heuristics or run-time analytics. For example, the magnet sensing logic 199 may be configured in accordance with machine-learning techniques to access a data repository 192 with pre-stored data (e.g., experiential knowledge of previously determined combinations of locations magnetic element 127 located at the tip 123 with ECG signals) pertaining to different locations of the tip 123 within superior vena cava. Specifically, such an embodiment may include processing of a machine-learning model trained using the experiential knowledge, where the determined tip locations serve as input to the trained model and processing of the trained model results in a determination as to how closely the determined combinations of a location the magnetic element 127 at the tip 123 with the ECG signal correlate to locations of the tip 123 within the superior vena cava.

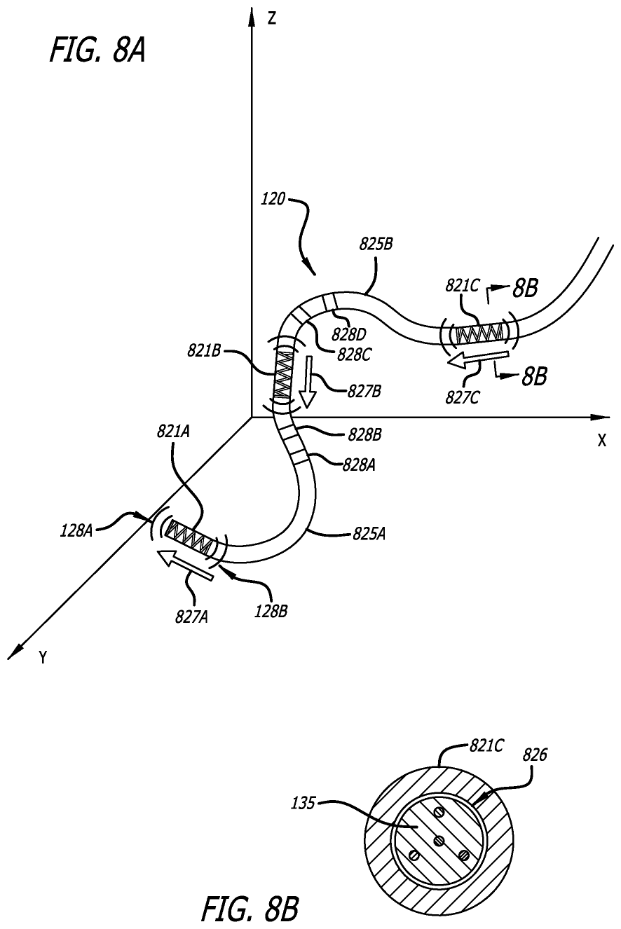

FIG. 8A illustrates the stylet 120 located within a space represented by a three-dimensional coordinate axis system, in accordance with some embodiments. In the illustrated embodiment, the stylet 120 includes three magnetic elements 821A-821C. The three-dimensional position and three-dimensional orientation of each of the magnetic elements 821A-821C is represented by the unit vectors 827A-827C, respectively. For example, the x-location, y-location and z-location of the unit vector 827A may define the position of the magnetic element 821A within the three-dimensional space. Similarly, directional components of the unit vector 827A in the x, y and z directions may define the orientation of the magnetic element 821A within the represented three-dimensional space.

A length segment 825A extends between magnetic elements 821A-821B and a length segment 825B extends between magnetic elements 821B-821C. According to one embodiment, the shape sensing logic 194 may determine the three-dimensional shape of length segments 825A, 825B of the stylet 120. In some embodiments, the magnetic elements 821A-821C may be spaced along the stylet 120 so that the lengths of segments 825A and 825B define a significant portion of the total length of the stylet 120. In such an embodiment, the magnetic elements 821A-821C may define a relatively gross position and orientation of the stylet 120. In other embodiments, the magnetic elements 821A-821C may be located in close proximity to each other, e.g., the lengths of segments 825A and 825B define a relatively short distal portion of the stylet 120 so that the magnetic elements 821A-821C may define a higher resolution of the position and orientation of the distal portion of the stylet 120. In such embodiments, the location of the magnetic elements 821A-821C may be defined in accordance with a defined use of the stylet 120. For example, a higher resolution of position and orientation of the distal portion of a central venous catheter may be advantageous for placement of the catheter tip with the lower third of the superior vena cava.

In some embodiments, the placement of a subset of shape sensing fiber sensors (fiber-based reflective gratings) may be located in close proximity to a magnetic element to define a region of the stylet 120 for enhanced position and orientation resolution. According to one example, in the illustrated embodiment, fiber sensors 828A, 828B are disposed immediately distal the magnetic element 821B and shape sensors 828A, 828B are disposed immediately proximal the magnetic element 821B. As such, the resolution of the physical state along the region of the stylet 120 between the shape sensors 828A, 828D may be greater adjacent the magnetic element 821B than adjacent the magnetic elements 821A, 821C. The higher resolution may result in enhanced placement accuracy of the stylet 120 within the vasculature. Other arrangements of shape sensors and magnetic elements to define discreet regions of higher and lower resolution are also contemplated.

FIG. 8B is a cross-sectional view of the stylet 120, in accordance with some embodiments. As show, the exemplary magnetic element 821C is formed of a hollow cylindrical shape defining an opening 826 or passageway extending therethrough. According one embodiment, the optical fiber core 135 passes through the opening 826. In some embodiments, the shape of magnetic element 821C may be representative of other magnetic elements 127 of FIGS. 1A, 1B.

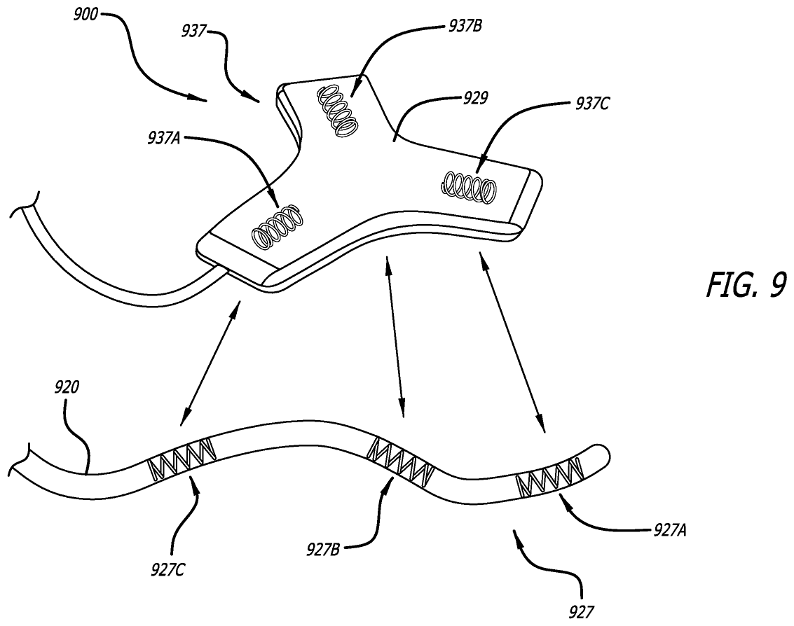

FIG. 9 illustrates a second embodiment of a magnetic location system 900 which may be employed with the medical instrument monitoring system 100 of FIGS. 1A-8, in accordance with some embodiments. The MLS 900 generally includes an elongate member 920 that may in some respects resemble the stylet 120 of FIG. 1A or the catheter 130 of FIG. 1B and a patient applied module 929 that may in some respects may resemble the magnet field sensor 129 of FIGS. 1A, 1B. The elongate member 920 includes a plurality of magnetic elements 927 including an exemplary subset of magnetic elements 927A-927C in the form of electro-magnetic elements electrically coupled with the console 110 (see FIGS. 1A, 1B), e.g., via wires (not shown) extending along the elongate member 920. The magnetic elements 927A-927C may be configured to operate as electro-magnets or magnetic field sensors. In some embodiments, one or more of the magnetic elements 927A-927C may be configured to operate only as electro-magnets defining magnetic fields. In other embodiments, one or more of the magnetic elements 927A-927C may be configured to operate only as magnet field sensors. In still other embodiments, one or more of the magnetic elements 927A-927C may be configured to selectively operate as either an electro-magnet or a magnetic field sensor.

In a similar fashion, the patient applied module 929 includes a plurality of magnetic elements 937 including an exemplary subset of magnetic elements 937A-937C in the form of electro-magnetic elements. The magnetic elements 937A-937C may be configured to operate as electro-magnets or magnetic field sensors. In some embodiments, one or more of the magnetic elements 937A-937C may be configured to operate only as electro-magnets defining magnetic fields. In other embodiments, one or more of the magnetic elements 937A-937C may be configured to operate only as magnet field sensors. In still other embodiments, one or more of the magnetic elements 937A-937C may be configured to selectively operate as either an electro-magnet or a magnetic field sensor.

According to one mode of operation, the magnetic elements 937A-937C are configured to operate as magnetic field sensors and magnetic elements 927A-927C are configured to operate as electro-magnets. As such, the MLS 900 determines the location and orientation of the elongate member 920 via magnetic fields defined by the magnetic elements 927A-927C and sensed by the magnetic elements 937A-937C. The magnetic elements 927A-927C may be operated (i.e., turned on, off, or otherwise modulated) according to one or more energizing modes. By way of one example, the magnetic elements 927A-927C may be singularly energized (i.e., one at a time). By way of another example, the magnetic elements 927A-927C may be modulated a different frequencies. In either case and other cases that may be contemplated, the MLS 900 may individually identify the magnetic fields defined by each of the magnetic elements 927A-927C, specifically.

Similarly, according to another mode of operation, the magnetic elements 927A-927C are configured to operate as magnetic field sensors and magnetic elements 937A-937C are configured to operate as electro-magnets. As such, the MLS 900 determines the location and orientation of the elongate member 920 via magnetic fields defined by the magnetic elements 937A-937C and sensed by the magnetic elements 927A-927C. The magnetic elements 937A-937C may be energized such that the MLS 900, via magnetic elements 927A-927C, may individually identify the magnetic fields defined by each of the magnetic elements 937A-937C, specifically. As may be contemplated by one of ordinary skill, other modes of operation may include any of the magnetic elements 927A-927C or the magnetic elements 937A-937C selectively operating as either an electro-magnet or a magnetic field sensor.

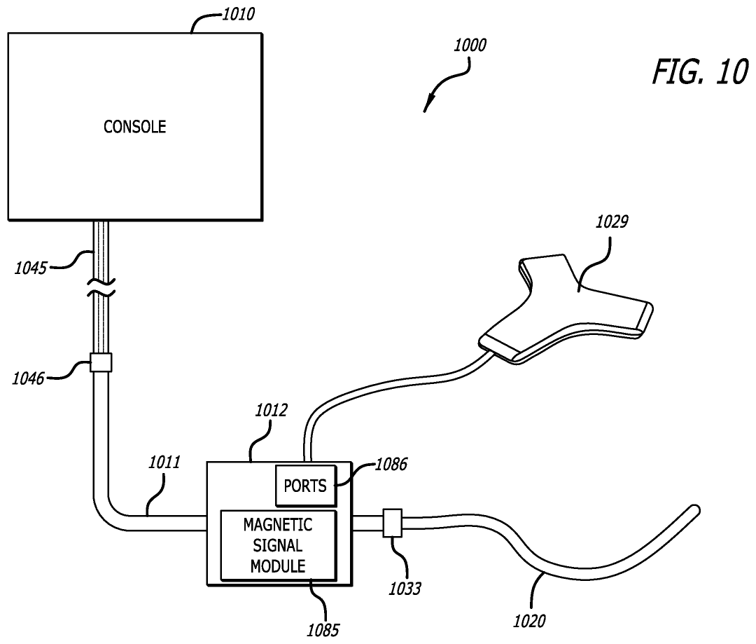

FIG. 10 illustrates another embodiment of a medical instrument monitoring system that can, in certain respects, resemble components of the medical instrument monitoring system 100 described in connection with FIGS. 1A-8. It will be appreciated that all the illustrated embodiments may have analogous features. Accordingly, like features are designated with like reference numerals, with a leading digit of “10.” For instance, the magnetic field sensor is designated as “129” in FIGS. 1A and 1B, and an analogous magnetic field sensor is designated as “1029” in FIG. 10. Relevant disclosure set forth above regarding similarly identified features thus may not be repeated hereafter. Moreover, specific features of the medical instrument monitoring system 100 and related components shown in FIGS. 1A and 1B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the medical instrument monitoring system of FIG. 10. Any suitable combination of the features, and variations of the same, described with respect to the medical instrument monitoring system 100 and components illustrated in FIG. 1A and 1B can be employed with the medical instrument monitoring system and components of FIG. 10, and vice versa

The medical instrument monitoring system 1000 includes a fiber optic patch cable 1011 coupled between the optical/electric connector 1046 and a console connector 1033 of the stylet 1020. In other embodiments, the system 1000 may include a catheter that may some respects resemble the catheter 130 for FIG. 1B in lieu of the stylet 1020. The patch cable 1011 includes a cable console 1012 and the cable console 1012 includes a magnetic signal module 1085 and ports 1086. The magnetic field sensor 1029 is electrically coupled with the cable console 1012 and the cable console 1012 is coupled with the console 1010 via the optical/electric connector 1046 and the interconnect 1045. The cable console 1012 includes connection ports 1086 for connection to the magnetic field sensor 1029. The magnetic signal module 1085 receives magnetic signals via the ports 1086 from the magnetic field sensor 1029 and converts the magnetic signals into magnetic data. In some embodiments, the magnetic data is electrically transferred to the console 1010 via the interconnect 1045. In other embodiments, the magnetic data may be wirelessly transmitted to the console 1010. The magnetic signal module 1085 may receive electrical power from the console 1010 or from a separate power supply (not shown).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A medical device system for detecting placement of a medical device within a patient body, the system comprising:
the medical device comprising:
an optical fiber having one or more of core fibers, each of the one or more core fibers including a plurality of fiber sensors distributed along a longitudinal length of a corresponding core fiber and each fiber sensor of the plurality of fiber sensors being configured to (i) reflect a light signal of a different fiber spectral width based on received incident light, and (ii) change a characteristic of the reflected light signal based on strain experienced by the optical fiber; and
a plurality of magnetic elements disposed along a longitudinal length of the medical device, each magnetic element defining a magnetic field configured to indicate a position of the magnetic element in three-dimensional space;
a magnetic field sensor configured to:
detect one or more magnetic fields defined by one or more of the plurality of magnetic elements, and
provide electrical signals in accordance with the detection of the one or more magnetic fields; and
a console including one or more processors and a non-transitory computer-readable medium having stored thereon logic, when executed by the one or more processors, causes operations including:
providing an incident light signal to the optical fiber;
receiving reflected light signals of different spectral widths of the incident light by one or more of the plurality of fiber sensors;
processing the reflected light signals associated with the one or more of core fibers to determine a physical state of the optical fiber;
receiving the electrical signals from the magnetic field sensor;
processing the electrical signals to determine the positions of the one or more of the plurality of magnetic elements with respect to the magnetic field sensor; and
combining the physical state of the medical device with the positions of the one or more of the plurality of magnetic elements to determine at least one of a position, a shape, and an orientation of the medical device within the patient body.

2. The system according to claim 1, wherein each magnetic element is longitudinally shaped having a magnetic pole at each end, the magnetic poles defining a magnetic field in accordance with an orientation of the magnetic element.

3. The system according to claim 2, wherein a longitudinal axis of each magnetic element is aligned with a longitudinal axis of the medical device.

4. The system according to claim 2, wherein the operations further include processing the electrical signals to determine an orientation of each of the magnetic elements with respect to the magnetic field sensor.

5. The system according to claim 4, wherein the operations further include combining the positions of the magnetic elements with the orientations of the magnetic elements to determine at least one of the position, the shape, and the orientation of the medical device within the patient body.

6. The system according to claim 1, wherein each magnet is shaped of a hollow cylinder, and wherein the optical fiber is disposed within the hollow cylinder.

7. The system according to claim 1, wherein at least one of the plurality of magnetic elements is disposed at a distal tip of the medical device.

8. The system according to claim 1, wherein the magnetic field sensor is applied to a chest of the patient.

9. The system according to claim 1, wherein one or more of the plurality of the magnetic elements are permanent magnets.

10. The system according to claim 1, wherein one or more of the plurality of the magnetic elements are formed of a ferrous material.

11. The system according to claim 1, wherein one or more of the plurality of magnetic elements are electro-magnets.

12. The system according to claim 11, wherein one or more of the plurality of magnetic elements are configured to sense a magnetic field.

13. The system according to claim 11, wherein one or more of the plurality of magnetic elements are configured to selectively define a magnetic field, and sense a magnetic field.

14. The system according to claim 1, wherein the magnetic field sensor includes one or more magnetic elements configured to define a magnetic field.

15. The system according to claim 1, wherein the medical device is one of an introducer wire, a guidewire, a stylet, a stylet within a needle, a needle with the optical fiber inlayed into a cannula of the needle or a catheter with the optical fiber inlayed into one or more walls of the catheter.

16. The system according to claim 1, wherein each of the plurality of fiber sensors is a reflective grating, where each reflective grating alters its reflected light signal by applying a wavelength shift dependent on a strain experienced by the reflective grating.

17. The system according to claim 1, wherein the operations further include:
receiving an ECG signal from an ECG electrode disposed at the distal tip;
processing the ECG signal to determine a position of the ECG electrode within the superior vena cava; and
combining the ECG signal with the physical state of the medical device and the positions of the one or more of the plurality of magnetic elements to determine the position of the medical device within the patient body.

18. The system according to claim 1, wherein the magnetic field sensor is coupled to the patient body.

19-28. (canceled)
